(19) United States
(12) Patent Application Publication
(10) Pub. No.: US 2010/0191229 A1
(43) Pub. Date: Jul. 29, 2010

(54) Methods for Employing Intrastromal Corrections in Combination with Surface Refractive Surgery to Correct Myopic/Hyperopic Presbyopia

(76) Inventors: Josef F. Bille, Heidelberg (DE); Luis Antonio Ruiz, Bogota (CO); Frieder Loesel, Mannheim (DE)

Correspondence Address: NYDEGGER & ASSOCIATES, 348 OLIVE STREET, SAN DIEGO, CA, 92103, US

(21) Appl. No.: 12/360,753

(22) Filed: Jan. 27, 2009

(51) Int. Cl. A61F 9/01; A61B 18/18

(52) U.S. Cl. 606/5

(57) ABSTRACT

A system and method for correcting a vision defect (i.e. presbyopia) of a patient requires two laser units. A first laser unit is used to photoablate (i.e. remove) tissue from the cornea for the creation of a multi-focal cornea that simultaneously provides for both near and distance vision capabilities. A second laser unit can also be used to refine the shape of the cornea by weakening selected portions with LIOB. Together, the removal and weakening of corneal tissue are regulated to optimize the resultant near vision and distant vision capabilities of the patient.

### FIELD OF THE INVENTION

The present invention pertains generally to ophthalmic, laser surgical procedures. More particularly, the present invention pertains to surgical procedures for the correction of presbyopia. The present invention is particularly, but not exclusively, useful as a system and method for combining the removal of corneal tissue by photoablation, with an intrastromal redistribution of biomechanical stresses by Laser Induced Optical Breakdown (LIOB) to achieve a refractive correction for a presbyopic eye.

### BACKGROUND OF THE INVENTION

By definition, presbyopia is farsightedness caused by the loss of elasticity in the lens of an eye that occurs in middle and old age. Basically, due to a loss of accommodation, an individual with presbyopia has difficulty seeing objects clearly, when they are relatively close to the eyes. Despite this difficulty, the distant vision of an individual with presbyopia may remain substantially unaffected. Nevertheless, the correction of presbyopia typically requires creating a multi-focal capability for the eyes that will have consequences for both near and far vision.

When refractive surgery is used for the correction of presbyopia, creating a multi-focal cornea requires making two essentially different refractive corrections. One of these is primarily for near vision correction and is made on corneal tissue immediately surrounding the visual axis. The other is for the preservation or correction of distant vision, and is made on corneal tissue that extends outwardly from the periphery of the near vision correction. A consequence of these corrections is the creation of a so-called presbyopic cone in the cornea. Structurally, the presbyopic cone is characterized by a relatively steep surface gradient that occurs in an interface region between the two corrections.

As can be expected, the resultant size of the presbyopic cone is an important consideration in refractive surgery. On the one hand, the presbyopic cone needs to be sufficiently large in diameter to achieve an effective near vision correction. On the other hand, the size of the presbyopic cone should not be so large that it hinders light from entering the pupil of the eye, and thereby diminishes or interferes with the person's distant vision. Thus, a balance is required between the near and distant vision corrections. Specifically, this is done to insure that both corrections are optimized. To achieve this balance, consideration must be given to several facts. For one, each patient is anatomically different. For another, the required near/distant vision corrections for each patient will be different. And, also the surgical results from one patient to another can be expected to be different. The situation can become further complicated when other vision defects such as myopia, hyperopia or astigmatism also need correction.

It is well known that surgically reshaping the cornea with a laser beam in order to achieve a refractive correction can be accomplished in either of two different ways. In one case, exposed superficial corneal tissue can be removed by photoablation, such as by the well known LASIK or PRK procedures. In the other case, a refractive correction can be achieved by weakening stromal tissue in the cornea with LIOB. This weakening then causes a redistribution of biomechanical stresses in the stroma that reshapes the cornea of the eye for the desired refractive correction. Both procedures are effective, but can have their respective advantages and limitations.

In light of the above, it is an object of the present invention to provide a system and method for combining the removal of corneal tissue by photoablation, with an intrastromal redistribution of biomechanical stresses by Laser Induced Optical Breakdown (LIOB) to achieve a refractive correction for a presbyopic eye. Another object of the present invention is to provide a system and method for a laser surgical procedure wherein the removal of corneal tissue is balanced with a weakening of stromal tissue to optimize the resultant near vision and distant vision of a presbyopic patient. Yet another object of the present invention is to provide a system and method for surgically treating a presbyopic eye that is easy to use, simple to implement and comparatively cost effective.

### SUMMARY OF THE INVENTION

A system for correcting a vision defect in accordance with the present invention includes two different laser units. One, a first laser unit, is used for accomplishing photoablation of corneal tissue of an eye. The other, a second laser unit, is used for performing Laser Induced Optical Breakdown (LIOB) in the stroma of the eye. Preferably, the first laser unit is of a type well known in the pertinent art as an excimer laser, and it is used to perform well known procedures, such as PRK or LASIK, for the removal of corneal tissue. On the other hand, the second laser unit is preferably capable of creating a pulsed femtosecond laser beam that is capable of performing LIOB for the purpose of weakening, rather than removing, stromal tissue. The present invention recognizes that these different procedures can complement each other.

For purposes of the present invention, the visual defect of primary concern is presbyopia and its unique effect on near vision. Specifically, with presbyopia, although the distant vision of a patient may be generally satisfactory, his/her near vision is adversely affected by a diminished capability for accommodation. In such a case, the necessary refractive correction essentially requires the creation of a simultaneous multi-focal capability. In detail, this entails creating a first refractive correction for near vision and a second refractive correction for simultaneous distant vision. Moreover, these corrections involve tissue in respectively different parts of the cornea. Consequently, the surgical alterations on the respective tissues need to be balanced in order to optimize the resultant near vision correction with the resultant distant vision correction.

In the operation of the system of the present invention, tissue is removed from the cornea of an eye to create a multi-focal refractive correction. Specifically, this is accomplished by photo-ablating the corneal tissue with the first laser unit (e.g. an excimer laser). As noted above, this multi-focal correction actually includes two differently identifiable corrections. A first correction is centered on the visual axis of the eye to correct the patient's near vision. This first correction extends directly from the visual axis in a radial direction, to a generally circular periphery. Abutting the first correction at its periphery is a second correction. It is this second correction that provides correction (or stabilization) for the distant vision of the patient. The creation of these two corrections results in a sloped interface region having a gradient between the first and second corrections. The net anatomical effect of these two corrections is the creation of a so-called presbyopic-cone.

From a surgical perspective, the size (i.e. diameter) of the presbyopic-cone must necessarily be established relative to the pupil size of the patient. Specifically, this is done in order to optimize both the patient's near vision and his/her distant vision. Further, depending on the magnitude of the gradient in the interface region between the first and second corrections, this optimization of near/distant vision may require some additional refinements in the consequent shape of the cornea. For the present invention, all of this is done with the second laser unit.

Along with the removal of corneal tissue by photoablation, and as mentioned above, the present invention also envisions selectively weakening tissue in the stroma of the eye. Specifically, this is done by LIOB to balance the first correction with the second correction, and to thereby optimize the multi-focal correction. For the present invention, the photoablation and weakening of corneal tissue can be accomplished in the same procedure. It is also possible to perform the photoablation and the weakening of corneal tissue at different times. If so, it is most likely that the photoablation would precede the weakening by as much as several weeks.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic view of a system for the present invention positioned relative to a cross-section view of the anterior portion of an eye; and

FIG. 2 is a cross-section view of the eye shown in FIG. 1 with a representative indication of where tissue is removed by photoablation and where tissue is weakened by LIOB in accordance with the present invention for the correction of presbyopia.

### DESCRIPTION OF THE PREFERRED EMBODIMENTS

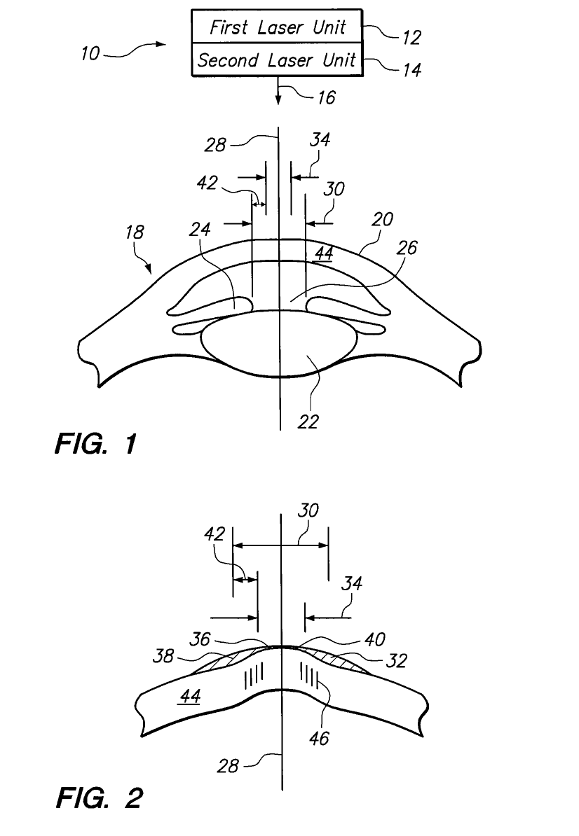

Referring initially to FIG. 1, a system for purposes of the present invention is shown and is generally designated 10. As shown, the system 10 includes a first laser unit 12 and a second laser unit 14. Further, each of the laser units 12 and 14 is shown respectively positioned to direct a laser beam along the beam path 16 toward an eye generally designated 18. Preferably, the first laser unit 12 is of a type well known in the pertinent art, such as an excimer laser. Specifically, the first laser unit 12 needs to be capable of photoablating (i.e. removing) tissue from the cornea 20 of the eye 18. On the other hand, the second laser unit 14 is preferably of a type that is capable of weakening tissue in the cornea 20 by performing Laser Induced Optical Breakdown (LIOB). Accordingly, the laser beam generated by the second laser unit 14 is preferably a pulsed laser beam having a sequence of individual pulses that are each less than about one picosecond in duration (i.e. a femtosecond laser).

Anatomically, in addition to the cornea 20, the anterior portion of the eye 18 (shown in FIG. 1) includes a lens 22. There is also an iris 24 that establishes the pupil 26 of the eye 18. Together, the cornea 20, the lens 22 and the iris 24 can be used to define the visual axis 28 of the eye 18. Depending on the dilation of the pupil 26, the pupil diameter 30 may vary.

As implied above, an operation of system 10 requires the coordinated activation of the first laser unit 12, with that of the second laser unit 14. More specifically, as best appreciated with reference to FIG. 2, the first laser unit 12 is employed for removing tissue from the cornea 20 by photoablation. Typically, this can be accomplished by such well-known surgical procedures as PRK or LASIK. For the specific case in which the visual defect being corrected is presbyopia, a volume 32 of tissue surrounding the visual axis 28 of the eye 18 is removed from the cornea 20. An exemplary volume 32 is shown cross-hatched in FIG. 2. In any event, the ablation volume 32 will be at a distance from the axis 28, and this distance is established to define the presbyopic diameter 34. Specifically, the presbyopic diameter 34 is determined by the greatest amount of tissue in the cornea 20 that can be used for near vision (i.e. a first vision correction) without unduly interfering with distant (far) vision of the eye 18 (i.e. a second vision correction).

The consequence of the above described photoablation is the creation of a so-called presbyopic-cone 36. As shown, the presbyopic-cone 36 has a surface that includes both unablated tissue around the visual axis 28, and newly exposed tissue in an interface region 38. More specifically, the top of the presbyopic-cone 36 (i.e. the unablated tissue), lies within a substantially circular periphery 40. Importantly, the periphery 40 is located at a predetermined distance from the visual axis 28 and is defined by the presbyopic diameter 34. The interface region 38 (i.e. ablated tissue) extends radially outward from the periphery 40 and is sloped with a gradient. Thus, a distance 42 that includes the interface region 38 is established. Specifically, the distance 42 is established as the difference between the presbyopic diameter 34 (required for near vision) and the pupil diameter 30 (required for distant vision). Thus, creation of the presbyopic-cone 36 effectively establishes a multi-focal capability for the eye 18. Stated differently, corneal tissue inside the periphery 40 will provide a near vision capability, and the remainder of the cornea 20 (i.e. corneal tissue outside the periphery 40) will provide a distant (far) vision capability. As envisioned for the present invention, this multi-focal capability will, however, most likely need some fine tuning.

When a multi-focal capability for the eye 18 is created as disclosed above, it can happen that additional aberrations may be introduced (induced) in the eye 18. This may be so, particularly, in the interface region 38. Further, because the size of the presbyopic diameter 34 (i.e. size of presbyopic cone 36) is selected relative to the size of the pupil diameter 30, with the object of optimizing both the near and distant vision of a patient, a balance between near and distant vision must be precisely preserved. For these purposes (i.e. aberration minimization and vision balance), a complementary weakening of the tissue in the stroma 44 of the cornea 20 can be used in accordance with the present invention to refine the reshaped cornea 20. Functionally, this weakening of tissue in the stroma 44 causes a redistribution of biomechanical stresses that responds to intraocular pressure in the eye 18 to reshape the cornea 20.

As envisioned for the present invention, a refinement of the cornea 20 to balance near and distant vision capabilities, and to minimize or eliminate induced aberrations, is accomplished using the second laser unit 14. Specifically, a pattern of incisions 46 can be made in the stroma 44 with the second laser unit 14 that will minimize any additionally induced aberrations, and will further optimize the surgical result by providing more balance for the multi-focal refractions created by the surgery.

While the particular Methods for Employing Intrastromal Corrections in Combination with Surface Refractive Surgery to Correct Myopic/Hyperopic Presbyopia as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for correcting a vision defect of a patient which comprises the steps of:
removing tissue from the cornea of an eye to create a multi-focal refractive correction, wherein the eye defines a visual axis and the multi-focal refractive correction includes a first correction centered on the visual axis and extending therefrom to correct near vision of the patient, and a second correction surrounding the first correction with an interface region therebetween, with the second correction extending from the first correction to correct distant vision of the patient; and
weakening tissue in the stroma of the eye to balance the first correction with the second correction to optimize the multi-focal correction.

2. A method as recited in claim 1 wherein the removing step is accomplished by photoablation of the corneal tissue.

3. A method as recited in claim 2 wherein the removing step is accomplished using a surgical procedure selected from the group consisting of LASIK and PRK.

4. A method as recited in claim 1 wherein the weakening step is accomplished by Laser Induced Optical Breakdown (LIOB).

5. A method as recited in claim 4 wherein the weakening step alters the first correction and the second correction of the multi-focal correction.

6. A method as recited in claim 1 wherein the removing step is accomplished using an excimer laser.

7. A method as recited in claim 1 wherein the weakening step is accomplished using a pulsed femtosecond laser beam.

8. A method as recited in claim 1 wherein the vision defect is presbyopia.

9. A method as recited in claim 8 wherein the vision defect includes at least one additional vision defect.

10. A method as recited in claim 9 wherein the additional vision defect is selected from a group consisting of myopia, hyperopia and astigmatism.

11. A method as recited in claim 1 wherein the removing step is accomplished before the weakening step, with a predetermined time interval therebetween.

12. A method as recited in claim 11 wherein the predetermined time interval is greater than approximately two weeks.

13. A method as recited in claim 1 wherein the removing step and the weakening step are accomplished substantially simultaneously.

14. A method for combining corneal tissue removal with an intrastromal redistribution of biomechanical stresses to achieve a predetermined refractive correction for an eye, the method comprising the steps of:
ablating selected corneal tissue in the eye;
performing Laser Induced Optical Breakdown (LIOB) on selected stromal tissue in the eye; and
regulating the ablating step with the performing step to balance a near vision requirement with a distant vision requirement during achievement of the predetermined refractive correction of the eye.

15. A method as recited in claim 14 wherein the ablating step is accomplished using an excimer laser and the performing step is accomplished using a pulsed femtosecond laser beam.

16. A method as recited in claim 14 wherein the eye defines a visual axis, and wherein the ablating step, the performing step and the regulating step, in combination, create a multi-focal correction including a first correction centered on the visual axis and extending therefrom to correct near vision of the patient, and a second correction surrounding the first correction with an interface region therebetween, with the second correction extending from the first correction to correct distant vision of the patient.

17. A method as recited in claim 14 wherein the vision defect is presbyopia.

18. A method as recited in claim 14 wherein the ablating step is accomplished before the performing step, with a predetermined time interval therebetween.

19. A method as recited in claim 18 wherein the predetermined time interval is greater than approximately two weeks.

20. A system for correcting a vision defect of a patient which comprises:
a first laser unit for removing tissue from the cornea of an eye to create a multi-focal refractive correction, wherein the eye defines a visual axis and the multi-focal refractive correction includes a first correction centered on the visual axis and extending therefrom to correct near vision of the patient, and a second correction surrounding the first correction with an interface region therebetween, with the second correction extending from the first correction to correct distant vision of the patient; and
a second laser unit for weakening tissue in the stroma of the eye to balance the first correction with the second correction to optimize the multi-focal correction.
